SEQUENCE LISTING (12 sequences)

SEQ ID NO: 1 - DNA, 4150 bp, Homo sapiens
cttgaatcttggggcaggaactcagaaaacttccagcccgggcagcgcgcgcttggtgcaagactcaggagctagcagcccgtccccctccgactctccggtgccgccgctgcctgctcccgccaccctaggaggcgcggtgccacccactactctgtcctctgcctgtgctccgtgcccgaccctatcccggcggagtctccccatcctcctttgctttccgactgcccaaggcactttcaatctcaatctcttctctctctctctctctctctctctctctctctctctctctctctctctctctctcgcagggtggggggaagaggaggaggaattctttccccgcctaacatttcaagggacacaattcactccaagtctcttccctttccaagccgcttccgaagtgctcccggtgcccgcaactcctgatcccaacccgcgagaggagcctctgcgacctcaaagcctctcttccttctccctcgcttccctcctcctcttgctacctccacctccaccgccacctccacctccggcacccacccaccgccgccgccgccaccggcagcgcctcctcctctcctcctcctcctcccctcttctctttttggcagccgctggacgtccggtgttgatggtggcagcggcggcagcctaagcaacagcagccctcgcagcccgccagctcgcgctcgccccgccggcgtccccagccctatcacctcatctcccgaaaggtgctgggcagctccggggcggtcgaggcgaagcggctgcagcggcggtagcggcggcgggaggcaggatgagcgcacgcggtgagggcgcggggcagccgtccacttcagcccagggacaacctgccgccccagcgcctcagaagagaggacgcggccgccccaggaagcagcagcaagaaccaaccggtgagccctctcctaagagacccaggggaagacccaaaggcagcaaaaacaagagtccctctaaagcagctcaaaagaaagcagaagccactggagaaaaacggccaagaggcagacctaggaaatggccacaacaagttgttcagaagaagcctgctcaggaggaaactgaagagacatcctcacaagagtctgccgaagaggactagggggcgccaacgttcgatttctacctcagcagcagttggatcttttgaagggagaagacactgcagtgaccacttattctgtattgccatggtctttccactttcatctggggtggggtggggtggggtgggggaggggggggtggggtggggagaaatcacataaccttaaaaaggactatattaatcaccttctttgtaatcccttcacagtcccaggtttagtgaaaaactgctgtaaacacaggggacacagcttaacaatgcaacttttaattactgttttcttttttcttaacctactaatagtttgttgatctgataagcaagagtgggcgggtgagaaaaaccgaattgggtttagtcaatcactgcactgcatgcaaacaagaaacgtgtcacacttgtgacgtcgggcattcatataggaagaacgcggtgtgtaacactgtgtacacctcaaataccaccccaacccactccctgtagtgaatcctctgtttagaacaccaaagataaggactagatactactttctctttttcgtataatcttgtagacacttacttgatgatttttaactttttatttctaaatgagacgaaatgctgatgtatcctttcattcagctaacaaactagaaaaggttatgttcatttttcaaaaagggaagtaagcaaacaaatattgccaactcttctatttatggatatcacacatatcagcaggagtaataaatttactcacagcacttgttttcaggacaacacttcattttcaggaaatctacttcctacagagccaaaatgccatttagcaataaataacacttgtcagcctcagagcatttaaggaaactagacaagtaaaattatcctctttgtaatttaatgaaaaggtacaacagaataatgcatgatgaactcacctaattatgaggtgggaggagcgaaatctaaatttcttttgctatagttatacatcaatttaaaaagcaaaaaaaaaaaaggggggggcaatctctctctgtgtctttctctctctctcttcctctccctctctcttttcattgtgtatcagtttccatgaaagacctgaataccacttacctcaaattaagcatatgtgttacttcaagtaatacgttttgacataagatggttgaccaaggtgcttttcttcggcttgagttcaccatctcttcattcaaactgcacttttagccagagatgcaatatatccccactactcaatactacctctgaatgttacaacgaatttacagtctagtacttattacatgctgctatacacaagcaatgcaagaaaaaaacttactgggtaggtgattctaatcatctgcagttctttttgtacacttaattacagttaaagaagcaatctccttactgtgtttcagcatgactatgtatttttctatgtttttttaattaaaaatttttaaaatacttgtttcagcttctctgctagatttctacattaacttgaaaattttttaaccaagtcgctcctaggttcttaaggataattttcctcaatcacactacacatcacacaagatttgactgtaatatttaaatattaccctccaagtctgtacctcaaatgaattctttaaggagatggactaattgacttgcaaagacctacctccagacttcaaaaggaatgaacttgttacttgcagcattcatttgttttttcaatgtttgaaatagttcaaactgcagctaaccctagtcaaaactatttttgtaaaagacatttgatagaaaggaacacgtttttacatacttttgcaaaataagtaaataataaataaaataaaagccaaccttcaaagaaacttgaagctttgtaggtgagatgcaacaagccctgcttttgcataatgcaatcaaaaatatgtgtttttaagattagttgaatataagaaaatgcttgacaaatattttcatgtattttacacaaatgtgatttttgtaatatgtctcaaccagatttattttaaacgcttcttatgtagagtttttatgcctttctctcctagtgagtgtgctgactttttaacatggtattatcaactgggccaggaggtagtttctcatgacggcttttgtcagtatggcttttagtactgaagccaaatgaaactcaaaaccatctctcttccagctgcttcagggaggtagtttcaaaggccacatacctctctgagactggcagatcgctcactgttgtgaatcaccaaaggagctatggagagaattaaaactcaacattactgttaactgtgcgttaaataagcaaataaacagtggctcataaaaataaaagtcgcattccatatctttggatgggccttttagaaacctcattggccagctcataaaatggaagcaattgctcatgttggccaaacatggtgcaccgagtgatttccatctctggtaaagttacacttttatttcctgtatgttgtacaatcaaaacacactactacctcttaagtcccagtatacctcatttttcatactgaaaaaaaaagcttgtggccaatggaacagtaagaacatcataaaatttttatatatatagtttatttttgtgggagataaattttataggactgttctttgctgttgttggtcgcagctacataagactggacatttaacttttctaccatttctgcaagttaggtatgtttgcaggagaaaagtatcaagacgtttaactgcagttgactttctccctgttcctttgagtgtcttctaactttattctttgttctttatgtagaattgctgtctatgattgtactttgaatcgcttgcttgttgaaaatatttctctagtgtattatcactgtctgttctgcacaataaacataacagcctctgtgatccccatgtgttttgattcctgctctttgttacagttccattaaatgagtaataaagtttggtcaaaacagaaaaaaaaaaa

SEQ ID NO: 2 - protein, 58 aa, Artificial Sequence
  Synthetic peptide
VTELLYRMKRAETYCPRPLLAIHPTEARHKQKIVAPVKQLLNFDLLKLAGDVESNPGP

SEQ ID NO: 3 - protein, 25 aa, Artificial Sequence
  Synthetic peptide
DGFCILYLLLILLMRSGDVETNPGP

SEQ ID NO: 4 - protein, 20 aa, Artificial Sequence
  Synthetic peptide
LLCFMLLLLLSGDVELNPGP

SEQ ID NO: 5 - protein, 20 aa, Artificial Sequence
  Synthetic peptide
HHFMFLLLLLAGDIELNPGP

SEQ ID NO: 6 - protein, 20 aa, Artificial Sequence
  Synthetic peptide
WFLVLLSFILSGDIEVNPGP

SEQ ID NO: 7 - protein, 20 aa, Artificial Sequence
  Synthetic peptide
KNCAMYMLLLSGDVETNPGP

SEQ ID NO: 8 - protein, 20 aa, Artificial Sequence
  Synthetic peptide
MVISQLMLKLAGDVEENPGP

SEQ ID NO: 9 - protein, 22 aa, Artificial Sequence
  Synthetic peptide
GSGATNFSLLKQAGDVEENPGP

SEQ ID NO: 10 - protein, 21 aa, Artificial Sequence
  Synthetic peptide
GSGEGRGSLLTCGDVEENPGP

SEQ ID NO: 11 - protein, 23 aa, Artificial Sequence
  Synthetic peptide
GSGQCTNYALLKLAGDVESNPGP

SEQ ID NO: 12 - protein, 8 aa, Artificial Sequence
  Synthetic peptide
  misc_feature (2)..(2): Xaa can be any naturally occurring amino acid
  misc_feature (4)..(4): Xaa can be any naturally occurring amino acid
DXEXNPGP

(19) United States
(12) Patent Application Publication
(10) Pub. No.: US 2019/0099451 A1
(43) Pub. Date: Apr. 4, 2019

(54) RETROVIRAL CONSTRUCT HARBORING A LET-7 INSENSITIVE NUCLEIC ACID ENCODING HMGA2 AND METHODS OF USE THEREOF

(71) Applicant: St. Jude Children's Research Hospital, Memphis, TN (US)

(72) Inventors: Sheng Zhou, Memphis, TN (US); Brian Sorrentino, Memphis, TN (US); Melissa Bonner, Natick, MA (US)

(21) Appl. No.: 16/082,359

(22) PCT Filed: Mar. 8, 2017
(86) PCT No.: PCT/US17/21257

§ 371 Date: Sep. 5, 2018

Related U.S. Application Data
(60) Provisional application No. 62/305,794, filed on Mar. 9, 2016.

(51) Int. Cl. A61K 35/30; A61K 38/17; C12N 15/85; C12N 15/62; A61K 35/28; C12N 5/0789

(52) U.S. Cl. CPC A61K 35/30; A61K 38/17; C12N 15/85; C12N 2740/10041; A61K 35/28; C12N 5/0647; C12N 15/62

(57) ABSTRACT

A retroviral construct harboring nucleic acids encoding a high mobility group AT-hook 2 (HMGA2) protein and lacking let-7 binding sites is described as are methods of using the retroviral vector to increase the efficacy and in vivo expansion of transduced cells in gene therapy applications.

### INTRODUCTION

This application claims the benefit of priority of U.S. Provisional Application No. 62/305,794, filed Mar. 9, 2016, the content of which is incorporated herein by reference in its entirety.

This invention was made with government support under contract number P01 HL 53749 awarded by the National Institutes of Health. The government has certain rights in the invention.

### BACKGROUND

In a lentiviral vector-based gene therapy trial for the treatment of β-thalassemia, a transplanted patient displayed a dominant myeloid cell clone harboring an integrated vector copy within the gene encoding HMGA2 (Cavazzana-Calvo, et al. (2010) Nature 467:318-322). Vector integration triggered the fusion of the splice donor sequence of the third exon of HMGA2 with a cryptic splice acceptor sequence present within an insulator element inserted in the vector long terminal repeat (LTR). This splicing event caused activation of the viral polyadenylation signal in the lentiviral LTR and induced premature HMGA2 transcript termination. This aberrant mRNA, lacking let-7 miRNA binding sites, displayed a higher stability that in turn lead to increased protein levels. Although not demonstrated, the activation of HMGA2 was suggested to be causative of the clonal dominance (Cavazzana-Calvo, et al. (2010) Nature 467:318-322).

Based on these findings, the consequence of HMGA2 overexpression has been investigated. More specifically, transgenic mice expressing a murine HMGA2 cDNA with a truncation of its 3′-untranslated region (UTR) have been generated (Ikeda, et al. (2011) Blood 117:5860-5869; Arlotta, et al. (2000) J. Biol. Chem. 275:14394-14400; Fedele, et al. (2006) Cancer Cell 9:459-471). HMGA2 over-expression increases body size and the incidence of certain tumors (Arlotta, et al. (2000) J. Biol. Chem. 275:14394-14400; Fedele, et al. (2006) Cancer Cell 9:459-471). Further, hematopoietic cells from the transgenic mice showed a growth advantage over wild-type cells in competitive repopulation assays and serial bone marrow transplant experiments, indicating that forced expression of ΔHmga2 leads to a proliferative growth advantage in hematopoietic stem and progenitor cells (Ikeda, et al. (2011) Blood 117:5860-5869). It has further been shown that lentivirus-mediated overexpression of full-length HMGA2 elevates self-renewal activity in transplanted irradiated mice (Copley, et al. (2013) Nature Cell Biol. 15:916-25).

WO 2011/158967 further indicates that the expression of HMGA2 improves efficiency in the establishment of induced pluripotent stem (iPS) cells by regulating the expression of p16INK4a or p19ARF. This reference suggests introducing HMGA2 and/or a Lin28A mutant protein into somatic cells for establishing iPS cells.

### SUMMARY OF THE INVENTION

This invention is a retroviral construct and transduced cell (e.g., a hematopoietic stem cell) harboring a let-7 insensitive nucleic acid encoding high mobility group AT-hook 2 (HMGA2) protein and nucleic acids encoding one or more therapeutic agents, e.g., a therapeutic protein or nucleic acid. In some embodiments, the let-7 insensitive nucleic acid has a mutation or deletion of one or more let-7 binding sites.

A method for increasing the efficacy and in vivo expansion of cells transduced with a retroviral vector is also provided. This method involves introducing a let-7 insensitive nucleic acid encoding a HMGA2 protein into a retroviral vector and transducing cells, e.g., hematopoietic stem cells, with the retroviral vector encoding the HMGA2 protein to increase the efficacy and in vivo expansion of the cells. In one embodiment, the retroviral vector further includes nucleic acids encoding one or more therapeutic agents. In another embodiment, the let-7 insensitive nucleic acid includes a mutation or deletion of one or more let-7 binding sites.

This invention is also a method for treating a disease or condition by transducing cells (e.g., hematopoietic stem cells) with a retroviral vector having a let-7 insensitive nucleic acid encoding a HMGA2 protein and nucleic acids encoding one or more therapeutic agents and introducing the cells into a subject in need of treatment with the one or more therapeutic agents. In one embodiment, the one or more therapeutic agents comprise a therapeutic protein or a therapeutic nucleic acid. In another embodiment, the let-7 insensitive nucleic acid includes a mutation or deletion of one or more let-7 binding sites. In a further embodiment, the subject receives a reduced intensity or low dose myeloablative conditioning regime prior to introducing the transduced cells.

This invention also provides a genetically-modified hematopoietic stem cell harboring a genetic alteration incurred by genome editing and including a construct having a let-7 insensitive nucleic acid encoding a HMGA2 protein, wherein in certain embodiments, the let-7 insensitive nucleic acid includes a mutation or deletion of one or more let-7 binding sites. Methods for enhancing genome editing efficiency in hematopoietic stem cells and treating a disease or condition using the genetically-modified hematopoietic stem cell are also provided.

### BRIEF DESCRIPTION OF THE DRAWINGS

FIG. 1 depicts nucleic acids encoding the HMGA2 transcript (SEQ. ID NO:1). The open reading frame is depicted in upper case letters and let-7 binding sites in the 3′-untranslated region are underlined.

FIGS. 2A and 2B show the percentage of peripheral blood leukocytes showing HMGA2-GFP and mCherry markings in animal #16 (A10W016; FIG. 2A)) and animal #27 (A10W027; FIG. 2B).

FIGS. 3A and 3B show the levels of HMGA2-GFP and mCherry marking in CD3⁺ T cells, CD14⁺ myeloid cells, CD16⁺ NK cells CD20⁺ B cells (FIG. 3A) and platelets and granulocytes (FIG. 3B) from animal #16 (A10W016) and animal #27 (A10W027), indicating that expansion had occurred in pluripotent hematopoietic stem cells. Filled circles, % GFP; open circles, % mCherry.

FIG. 4 shows oligoclonal marking in both the GFP⁺ and mCherry⁺ cells in animal #16 (A10W016) and animal #27 (A10W027), with numerous clones contributing to the peripheral blood CD14⁺ compartment.

### DETAILED DESCRIPTION OF THE INVENTION

Gene therapy with integrative vectors can be used to treat many different kinds of human diseases, such as sickle cell anemia, immunodeficiencies, etc. One challenge is a relatively low transduction efficiency with these vectors in human long-term hematopoietic stem cells, which limits their effective use. While protocols have been developed in mouse models, such protocols are not effective with primate hematopoietic stem cells (HSCs). For example, while overexpression of homeobox B4 (HOXB4) in murine transplantation models has been shown to induce ex vivo expansion and self-renewal of HSCs, retroviral-mediated transduction of rhesus macaque HSCs with HOXB4 results in no substantial increase in HSC amplification (Larochelle, et al. (2009) J. Clin. Invest. 119(7):1952-63). Therefore, novel approaches for increasing transduction efficiency of stem cells must be demonstrated in a relevant animal model to demonstrate efficacy and safety for human use.

It has now been discovered that overexpression of a truncated form of human HMGA2 transcript by a retroviral vector can expand hematopoietic stem cells in transplanted Nemestrina Macaque, a highly relevant non-human primate model. More specifically, it was observed that by incorporating the truncated HMGA2 nucleic acids in a vector, the small number of hematopoietic stem cells that are initially transduced (about 1%) can progressively expand over a period of two years to 40-70% after transplant in recipient Nemestrina Macaque. In light of these results, the present invention provides a retroviral construct harboring nucleic acids encoding a truncated HMGA2 transcript for use in delivering therapeutic agents, such as gamma-globin encoding sequences, and achieving in vivo expansion of transduced hematopoietic stem cells thereby improving efficacy. This construct can be used for the modification of stem cells (e.g., hematopoietic stem and progenitor cells) that can be introduced into a subject in need thereof for the treatment of a variety of diseases including, but not limited to hemoglobinopathies such as sickle cell disease (SCD), Wiscott-Aldrich Syndrome (WAS), and X-linked severe combined immunodeficiency (SCIDXI), chronic granulomatous disease, beta-thalassemia, lysosomal storage diseases, and hemophilia disorders. Moreover, this construct can be directly administered to a subject to achieve in vivo transduction of the target cells (e.g., hematopoietic stem or progenitor cells) and thereby also effect a treatment of subjects in need thereof.

The high mobility group AT-hook 2 (HMGA2) protein is a member of the HMGA family of nonhistone chromatin proteins, which also includes HMGA1a, HMGA1b, and HMGA1c (Sgarra, et al. (2004) FEBS Lett. 574:1-8). Exons 1 to 3 of the HMGA2 gene encode DNA-binding AT-hook domains, which can modulate transcription by affecting the DNA conformation of specific AT-rich regulatory elements promoting transcriptional activity. Exon 4 acts as a linker, and exon 5 encodes the acidic C-terminal domain of the protein and the 3′ untranslated region (UTR) of the mRNA (Zhou, et al. (1996) Nucleic, Acids Res. 24(20):4071-4077; Chen, et al. (2010) Biochemistry 49(8):1590-1595). The HMGA2 protein is important in a wide spectrum of biologic processes, including cell proliferation, cell-cycle progression, apoptosis, and senescence (Fusco & Fedele (2007) Nat. Rev. Cancer. 7(12):899-910; Young & Narita (2007) Genes Dev. 21(9):1005-1009), and has been suggested to play a role in self-renewal and control of differentiation of embryonic stem (ES) cells (Li, et al. (2007) FEBS Lett. 581(18):3533-3537), cancer stem cells (Yu, et al. (2007) Cell 131(6):1109-1123), and neural stem cells (Nishino, et al. (2008) Cell 135(2):227-239).

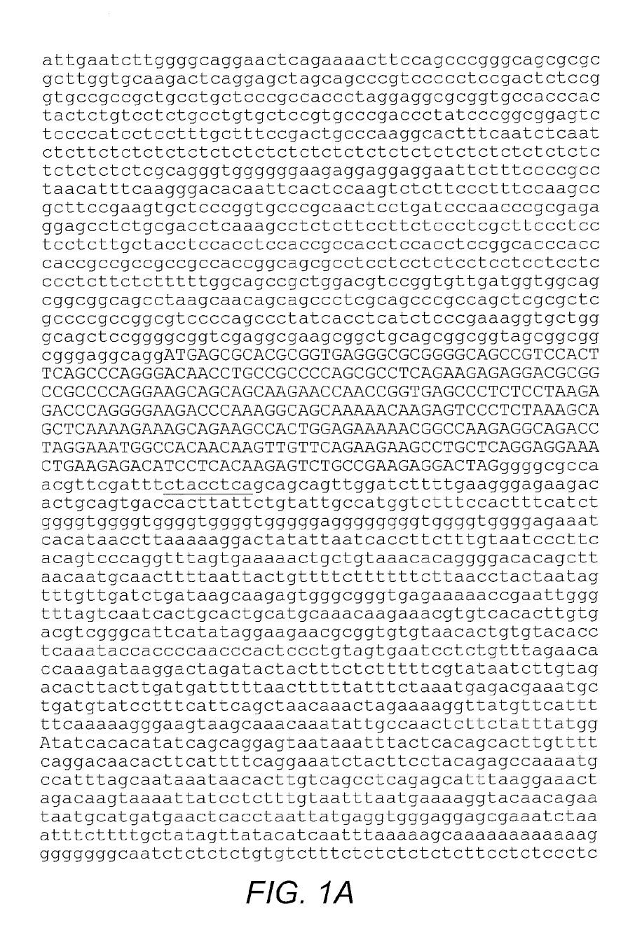

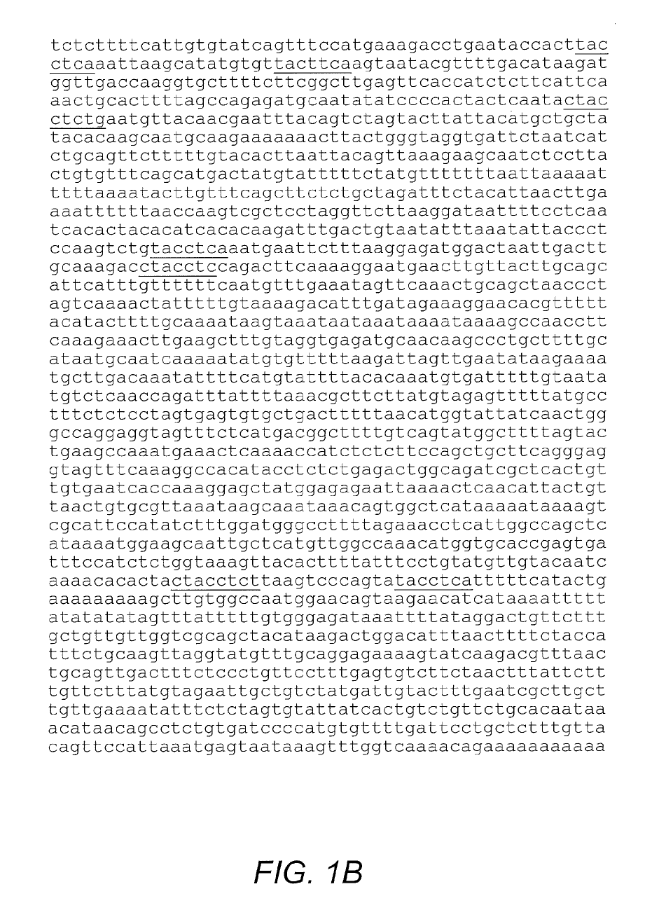

The HMGA2 transcript provided under GENBANK Accession No. NM_003483 is depicted in FIGS. 1A and 1B (SEQ ID NO:1), which show sequences complementary to the let-7-family of microRNAs (see underlined sequences in FIGS. 1A and 1B). Binding of the complementary sequences by let-7 miRNAs post-transcriptionally and negatively regulates HMGA2 mRNA and protein expression (Young & Narita (2007) Genes Dev. 21(9):1005-1009). In this respect, “a let-7 insensitive nucleic acid encoding HMGA2” refers to an HMGA2 transcript with mutations or deletions in one or more sequences complementary to the let-7-family. In some embodiments, a let-7 binding site is eliminated by one or more point mutations. In another embodiment, all or a portion of a let-7 binding site it deleted. As illustrated in FIGS. 1A and 1B, let-7 binding sites are located at nucleotides 1161-1169; 2248-2254; 2271-2277; 2397-2405; 2760-2766; 2809-2815; 3662-3669 or 3682-3688 of SEQ ID NO:1. In some embodiments, the HMGA2 transcript is truncated to remove at least nucleotides 1161 to 3688 of SEQ ID NO:1. In other embodiments, the HMGA2 transcript is truncated to remove at least nucleotides 1161 to 4150 of SEQ ID NO:1. Given that let-7 binding regulates HMGA2 expression, one or more let-7 binding sites can remain intact (i.e., not deleted or mutated) as a means to modulate HMGA2 levels in vivo. Therefore, in certain embodiments, 1, 2, 3, 4, 5, 6, or all 7 let-7 binding sites are mutated or deleted in the retroviral construct herein. In a further embodiment of this invention, a tamoxifen regulatable form of HMGA2 is used to control hematopoietic stem cell expansion pharmacologically.

As an alternative to HMGA2, it is contemplated that other genes in this same pathway can be overexpressed/repressed to achieve in vivo expansion of transduced hematopoietic stem cells and improve efficacy. For example, declines in stem cell function within aging tissues are partially caused by increasing p16Ink4a expression. In this respect, p16Ink4a deficiency partially rescues the decline in stem and progenitor cell function in the aging central nervous system and other tissues without affecting stem and progenitor cell function in young adult tissues (Janzen, et al. (2006) Nature 443:421-426; Krishnamurthy, et al. (2006) Nature 443:453-457; Molofsky, et al. (2006) Nature 443:448-452). Similarly, p19ARF expression also increases in aging tissues (Molofsky, et al. (2006) Nature 443:448-452). However, neither of these proteins is expressed in postnatal stem cells as expression is repressed by Bmi-1 and HMGA2 (Nishino, et al. (2008) Cell 135:227-239). Moreover, JunB has been suggested to promote p16Ink4a/p19ARF in the absence of HMGA2 (Nishino, et al. (2008) Cell 135:227-239). Accordingly, a retroviral construct for use in in vivo expansion of transduced hematopoietic stem cells can encode for an inhibitory RNA (e.g., siRNA or antisense RNA) that represses the expression of one or more of p16Ink4a, p19ARF or JunB. Alternatively, a retroviral construct for use in in vivo expansion of transduced hematopoietic stem cells can provide for the overexpression of Bmi-1 thereby repressing p16Ink4a/p19ARF.

In addition to a let-7 insensitive nucleic acid encoding HMGA2, the retroviral vector optionally co-expresses one or more therapeutic agents. A therapeutic agent is intended to include a protein (e.g., an enzyme or vaccine) or nucleic acid (e.g., siRNA, ribozymes, anti-sense, and other functional polynucleotides) that prevents, ameliorates, delays onset, lessens or treats a disease or condition of interest. In the case of hematopoietic stem cells, one will typically select a therapeutic agent that will confer a desirable function on such cells, including, for example, globin genes, hematopoietic growth factors, which include erythropoietin (EPO), the interleukins (such as Interleukin-1 (IL-1), Interleukin-2 (IL-2), Interleukin-(IL-3), Interleukin-6 (IL-6), Interleukin-12 (IL-12), etc.) and the colony-stimulating factors (such as granulocyte colony-stimulating factor, granulocyte/macrophage colony-stimulating factor, or stem-cell colony-stimulating factor), the platelet-specific integrin αIIbβ, multidrug resistance genes, the gp91 or gp 47 genes that are defective in patients with chronic granulomatous disease (CGD), antiviral genes rendering cells resistant to infections with pathogens such as human immunodeficiency virus, genes coding for blood coagulation factors VIII or IX which are mutated in hemophiliacs, ligands involved in T cell-mediated immune responses such as T cell antigen receptors, B cell antigen receptors (immunoglobulins) as well as combination of T and B cell antigen receptors alone or in combination with single chain antibodies such as ScFv, tumor necrosis factor (TNF), IL-2, IL-12, gamma interferon, CTLA4, B7 and the like, genes expressed in tumor cells such as Melana, MAGE genes (such as MAGE-1, MAGE-3), P198, P1A, gp100, etc.

In some embodiments, more than one therapeutic agent is delivered. For example, a retroviral construct expressing an RNAi targeted to beta-hemoglobin can repress or silence sickle-hemoglobin in patients with sickle cell anemia. The same retroviral construct can also express a normal hemoglobin molecule that has been codon-degenerated at the site targeted by the RNAi. In this way, erythroid cells expressing sickle globin can repress sickle globin expression, while expressing native hemoglobin and correct the genetic abnormality. The retroviral construct would be delivered into a stem cell population that would give rise to erythroid cells expressing hemoglobin that would eventually become red cells.

A principal application of the retroviral construct of the invention is to deliver desired therapeutic agents to hematopoietic cells for a number of possible reasons including, but not be limited to, the treatment of genetic disorders, cancers, myelosupression and neutropenias, infections such as AIDS, and the like. Exemplary genetic disorders of hematopoietic cells that that can be treated with the retroviral construct of this invention include, e.g., sickle cell anemia, thalassemias, hemaglobinopathies, Glanzmann thrombasthenia, lysosomal storage disorders (such as Fabry disease, Gaucher disease, Niemann-Pick disease, and Wiskott-Aldrich syndrome), severe combined immunodeficiency syndromes (SCID), as well as diseases resulting from the lack of systemic production of a secreted protein, for, example, coagulation factor VIII and/or IX. Exemplary cancers are those of hematopoietic origin, for example, arising from myeloid, lymphoid or erythroid lineages, or precursor cells thereof. Exemplary myeloid disorders include, but are not limited to, acute promyeloid leukemia (APML), acute myelogenous leukemia (AML) and chronic myelogenous leukemia (CML). Lymphoid malignancies which may be treated utilizing the retroviral construct of the invention include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas contemplated as candidates for treatment with the retroviral construct of the invention include, but are not limited to non-Hodgkin lymphoma and variants thereof, peripheral T-cell lymphomas, adult T-cell leukemia/lymphoma (ATL), cutaneous T-cell lymphoma (CTCL), large granular lymphocytic leukemia (LGF) and Hodgkin disease. Moreover, the retroviral construct of this invention can be used in the treatment of non-hematological diseases as well, including, e.g., X-linked adrenoleukodystrophy (ALD; Cartier, et al. (2009) Science 326(5954):818-23).

In the retroviral construct of, the invention, at least one promoter directs transcription of the let-7 insensitive nucleic acid encoding HMGA2 and nucleic acids encoding the one or more therapeutic agents. According to Some embodiments, the let-7 insensitive nucleic acid encoding HMGA2 and nucleic acids encoding the one or more therapeutic agent are independently expressed via different promoters, i.e., the nucleic acid encoding HMGA2 is operably linked to a first promoter and nucleic acids encoding the one or more therapeutic agents are operably linked to a second promoter (and optionally third or more promoters), which may be the same or different than the first promoter. In other embodiments, the nucleic acid encoding HMGA2 and the nucleic acids encoding the one or more therapeutic agents are co-expressed via a single promoter, i.e., the nucleic acid encoding HMGA2 and nucleic acids encoding the therapeutic agents are in tandem and operably linked to a single promoter. A coding nucleic acid is “operably linked” to a regulatory sequence (e.g., promoter) if the regulatory sequence is capable of exerting a regulatory effect on the coding sequence linked thereto. In other words, the promoter(s) of the invention is positioned so as to promote transcription of the messenger RNA from the nucleic acids encoding HMGA2 and the therapeutic agent.

The promoter(s) of the invention can be of genomic origin or synthetically generated. The promoters may or may not be associated with enhancers, wherein the enhancers may be naturally associated with the particular promoter or associated with a different promoter. A variety of promoters for use in hematopoietic stem cells have been described in the art. For example, numerous examples of elements/promoters of use in this invention are described in U.S. Pat. No. 8,748,169, incorporated herein by reference. The promoter can be constitutive or inducible, where induction is associated with the specific cell type, a specific level of maturation, or drug (e.g., tetracycline or doxorubicin).

The simultaneous or co-expression of HMGA2 and a therapeutic agent via a single promoter may be achieved by the use of an internal ribosomal entry site (IRES) or cis-acting hydrolase element. The term “internal ribosome entry site” or “IRES” defines a sequence motif that promotes attachment of ribosomes to that motif on internal mRNA sequences. Consequently, an mRNA containing an IRES sequence motif results in two translational products, one initiating from the 5′-end of the mRNA and the other by an internal translation mechanism mediated by the IRES. A number of IRES have been described and can be used in the nucleic acid construct of this invention. See, e.g., U.S. Pat. No. 8,192,984; WO 2010/119257; and US 2005/0112095.

A “cis-acting hydrolase element” or “CHYSEL” refers to a peptide sequence that causes a ribosome to release the growing polypeptide chain that it is being synthesizes without dissociation from the mRNA. In this respect, the ribosome continues translating and therefore produces a second polypeptide. Peptides such as the foot and mouth disease virus (FMDV) 2A sequence (VTELLYRMKRAETYC PRPLLAIHPTEARHKQKIVAPVKQLLNFDLLKLAGDVESNPGP, SEQ ID NO:2), sea urchin (Strongylocentrotus purpuratus) 2A sequence (DGFCILYLLLILLMRSGDVETNPGP, SEQ ID NO:3); Sponge (Amphimedon queenslandica) 2A sequence (LLCFMLLLLLSGDVELNPGP, SEQ ID NO:4; or HHFMFLLLLL AGDIELNPGP, SEQ ID NO:5); acorn worm (Saccoglossus kowalevskii) (WFLVLLSFILSGDIEVNPGP, SEQ ID NO:6) 2A sequence; amphioxus (Branchiostoma floridae) (KNCAMYMLLLSGDVETNPGP, SEQ ID NO:7; or MVISQLMLKLAGDVEENPGP, SEQ ID NO:8) 2A sequence porcine teschovirus-1 (GSGATNFSLLKQAGDVEENPGP, SEQ ID NO:9) 2A sequence; Thoseaasigna virus (GSGEGRGSLLTCGDVEENPGP, SEQ ID NO:10) 2A sequence; and equine rhinitis A virus (GSGQCTNYALLKLAGDVESNPGP, SEQ ID NO:11) 2A sequence are CHYSELs of use in this invention. In some embodiments, the 2A sequence is a naturally occurring or synthetic sequence that includes the 2A consensus sequence D-X-E-X-NPGP (SEQ ID NO:12), in which X is any amino acid residue.

For expression of HMGA2 and the therapeutic agent, the naturally occurring or endogenous transcriptional initiation region of the nucleic acid sequence encoding HMGA2 or the therapeutic agent can be used to initiate transcription of HMGA2 and the therapeutic agent in the target cell. Alternatively, an exogenous transcriptional initiation region can be used which allows for constitutive or inducible expression, wherein expression can be controlled depending upon the target cell, the level of expression desired, the nature of the target cell, and the like. Likewise, the termination region(s) of the construct may be provided by the naturally occurring or endogenous transcriptional termination region of the nucleic acids encoding HMGA2 or the therapeutic agent. Alternatively, the termination region may be derived from a different source.

As used herein, the term “retroviral vector” or “retroviral construct” refers to an integrative vector system or construct derived from the Retroviridae family of viruses. In certain embodiments, the retroviral vector or construct is a spumaviral vector or construct. Spumaviruses or foamy viruses are unique retroviruses that have evolved means for efficient transmission and infection of their hosts without pathology. Foamy virus vectors have several unique properties that make them well-suited for therapeutic gene transfer including a desirable safety profile, a broad tropism, a large transgene capacity, and the ability to persist in quiescent cells. In addition, they mediate efficient and stable gene transfer to hematopoietic stem cells (HSCs). These attributes have led to the development of vectors derived from several foamy viruses including the prototypic foamy virus (PFV), simian foamy virus type 1 (SFV-1, macaque), and feline foamy virus (FFV). See, e.g., Trobridge (2009) Expert Opin. Biol. Ther. 9:1427-36; Olszko & Trobridge (2013) Viruses 5:2585-2600.

In other embodiments, the retroviral vector or construct is a lentiviral vector or construct. The term “lentiviral vector” or “lentiviral construct” has its general meaning in the art, see Naldini, et al. (1996 and 1998); Zufferey et al., (1997); Dull et al., (1998), Ramezani et al., (2000), U.S. Pat. Nos. 5,994,136; 6,013,516; 6,165,782; 6,207,455; 6,218,181; 6,218,186; and 6,277,633. In general, these vectors are plasmid-based or virus-based, and are configured to carry the essential sequences for incorporating foreign nucleic acid, for selection and for transfer of the nucleic acid into a host cell. Lentiviral vectors are of particular use in the present invention as they generally do not integrate into cellular oncogenes such as LMO2, PRDM1 or EVI1, they give superior transduction efficiency in hematopoietic stem cells and can be produced at high titers. Any suitable lentiviral vector can be used including primate and non-primate lentiviruses. Specific examples of species, include, but are not limited to, e.g., HIV-1 (including subspecies, clades, or strains, such as A, B, C, D, E, F, and G, R5 and R5X4 viruses, etc.), HIV-2 (including subspecies, clades, or strains, such as, R5 and R5X4 viruses, etc.), simian immunodeficiency virus (SIV), simian/human immunodeficiency virus (SHIV), feline immunodeficiency virus (FIV), bovine immunodeficiency virus (BIV), murine stem cell virus (MSCV), caprine-arthritis-encephalitis virus, Jembrana disease virus, ovine lentivirus, visna virus, and equine infectious anemia virus. Genomic sequence for such viruses are widely available, e.g., HIV-1 (NC_001802), HIV-2 (NC_001722), SIV (NC_001549), SIV-2 (NC_004455), Caprine arthritis-encephalitis virus (NC_001463), Simian-Human immunodeficiency virus (NC_001870), FIV (NC_001482), MSCV (AX823827; WO 03/070958), Jembrana disease virus (NC_001654), ovine (NC_001511), Visna virus (NC_001452), Equine infectious anemia virus (NC_001450), and BIV (NC_001413).

For therapeutic applications, it is desirable that the retroviral construct includes one or more of the following components: an expression cassette encoding a therapeutic agent (e.g., an anti-sickling human β-globin); substitution of the HIV LTR with a CMV promoter to yield a higher titer vector without the inclusion of the HIV TAT protein during packaging; a self-inactivating (SIN) LTR configuration; an (optional) insulator element (e.g., FB); a packaging signal (e.g., W); a Rev Responsive Element (RRE) to enhance nuclear export of unspliced vector RNA; a central polypurine tract (cPPT) to enhance nuclear import of vector genomes; and/or a post-transcriptional regulatory element (PRE) to enhance vector genome stability and to improve vector titers (e.g., WPRE).

TAT-Independent and Self-Inactivating Lentiviral Vectors.

In certain embodiments, the lentiviral construct described herein includes a self-inactivating (SIN) configuration to increase the biosafety of the lentiviral construct. SIN vectors are ones in which the production of full-length vector RNA in transduced cells is greatly reduced or abolished altogether. This feature minimizes the risk that replication-competent recombinants (RCRs) will emerge. Furthermore, it reduces the risk that that cellular coding sequences located adjacent to the vector integration site will be aberrantly expressed. Furthermore, a SIN design reduces the possibility of interference between the LTR and the promoter(s) that is driving the expression of the transgene(s). SIN lentiviral, constructs can often permit full activity of the internal promoter.

As viral transcription starts at the 3′ end of the U3 region of the 5′-LTR, those sequences are not part of the viral mRNA and a copy thereof from the 3′-LTR acts as template for the generation of both LTR's in the integrated provirus. If the 3′ copy of the U3 region is altered in a retroviral vector construct, the vector RNA is still produced from the intact 5′-LTR in producer cells, but cannot be regenerated in target cells. Transduction of such a vector results in the inactivation of both LTR's in the progeny virus. Thus, the retrovirus is self-inactivating (SIN). The SIN design is described in detail in Zufferey, et al. (1998) J. Virol. 72(12):9873-9880, and U.S. Pat. No. 5,994,136. Additional SIN designs are described in US 2003/0039636.

In some embodiments, lentiviral sequences are removed from the LTRs and are replaced with comparable sequences from a non-lentiviral retrovirus, thereby forming hybrid LTRs. In particular, the lentiviral R region within the LTR can be replaced in whole or in part by the R region from a non-lentiviral retrovirus. In certain embodiments, the lentiviral TAR sequence, a sequence which interacts with TAT protein to enhance viral replication, is removed, preferably in whole, from the R region. The TAR sequence is then replaced with a comparable portion of the R region from a non-lentiviral retrovirus, thereby forming a hybrid R region. The LTRs can be further modified to remove and/or replace with non-lentiviral sequences all or a portion of the lentiviral U3 and U5 regions. Accordingly, in certain embodiments, the SIN configuration provides a retroviral LTR composed of a hybrid lentiviral R region that lacks all or a portion of its TAR sequence, thereby eliminating any possible activation by TAT, wherein the TAR sequence or portion thereof is replaced by a comparable portion of the R region from a non-lentiviral retrovirus, thereby forming a hybrid R region.

Suitable lentiviruses from which the R region can be derived include, for example, HIV (HIV-1 and HIV-2), EIV, SIV and FIV. Suitable retroviruses from which non-lentiviral sequences can be derived include, for example, MoMSV, MoMSV, Friend, MSCV, RSV and Spumaviruses. In one illustrative embodiment, the lentivirus is HIV and the non-lentiviral retrovirus is MoMSV.

In another embodiment, the left (5′) LTR further includes a promoter sequence upstream from the hybrid R region. Preferred promoters are non-lentiviral in origin and include, for example, the U3 region from a non-lentiviral retrovirus (e.g., the MoMSV U3 region). Examples of such a left (5′) LTR are described in US 2003/0039636.

In another illustrative embodiment, the right (3′) LTR further includes a modified (e.g., truncated) lentiviral U3 region upstream from the hybrid R region. The modified lentiviral U3 region can include the att sequence, but lack any sequences having promoter activity, thereby causing the vector to be SIN in that viral transcription cannot go beyond the first round of replication following chromosomal integration. In a particular embodiment, the modified lentiviral U3 region upstream from the hybrid R region is composed of the 3′ end of a lentiviral (e.g., HIV) U3 region up to and including the lentiviral U3 att site. Examples of such a right (3′) LTR are described in US 2003/0039636.

In the case of HIV-based lentiviral vectors, it has been discovered that such vectors tolerate significant U3 deletions, including the removal of the LTR TATA box (e.g., deletions from −418 to −18), without significant reductions in vector titers. These deletions render the LTR region substantially transcriptionally inactive in that the transcriptional ability of the LTR in reduced to about 90% or lower. It has also been demonstrated that the trans-acting function of Tat becomes dispensable if part of the upstream LTR in the transfer vector construct is replaced by constitutively active promoter sequences (see, e.g., Dull, et al. (1998) J. Virol. 72(11):8463-8471).

It will be recognized that the CMV promoter typically provides a high level of non-tissue specific expression. Other promoters with similar constitutive activity include, but are not limited to the RSV promoter, and the SV40 promoter. Mammalian promoters such as the beta-actin promoter, ubiquitin C promoter, elongation factor α promoter, tubulin promoter, etc., may also be used.

As indicated above, in certain embodiments, the LTR transcription is reduced by about 95% to about 99%. In certain embodiments LTR may be rendered at least about 90%, at least about 91%, at least about 92%, at least about 93%, at least about 94%, at least about 95%, at least about 96%, at least about 97%, at least about 98%, or at least about 99% transcriptionally inactive.

Insulator Element.

To further enhance biosafety insulators are inserted into the lentiviral construct described herein. Insulators are DNA sequence elements present throughout the genome. They bind proteins that modify chromatin and alter regional gene expression. The placement of insulators in the vectors described herein offer various potential benefits including, inter alia, shielding of the vector from positional effect variegation of expression by flanking chromosomes (i.e., barrier activity); and shielding flanking chromosomes from insertional trans-activation of gene expression by the vector (enhancer blocking). Thus, insulators can help to preserve the independent function of genes or transcription units embedded in a genome or genetic context in which their expression may otherwise be influenced by regulatory signals within the genome or genetic context (see, e.g., Burgess-Beusse, et al. (2002) Proc. Natl. Acad. Sci. USA 99:16433; Zhan, et al. (2001) Hum. Genet. 109:471). In the present context, insulators may contribute to protecting lentivirus-expressed sequences from integration site effects, which may be mediated by cis-acting elements present in genomic DNA and lead to deregulated expression of transferred sequences. In various embodiments, a lentiviral construct is provided in which an insulator sequence is inserted into one or both LTRs or elsewhere in the region of the vector that integrates into the cellular genome.

The first and best characterized vertebrate chromatin insulator is located within the chicken β-globin locus control region. This element, which contains a DNase-I hypersensitive site-4 (cHS4), appears to constitute the 5′ boundary of the chicken β-globin locus (Prioleau, et al. (1999) EMBO J. 18:4035-4048). A 1.2-kb fragment containing the cHS4 element displays classic insulator activities, including the ability to block the interaction of globin gene promoters and enhancers in cell lines (Chung, et al. (1993) Cell 74:505-514), and the ability to protect expression cassettes in Drosophila, transformed cell lines (Pikaart, et al. (1998) Genes Dev. 12:2852-2862), and transgenic mammals (Wang, et al. (1997) Nat. Biotechnol. 15:239-243; Taboit-Dameron, et al. (1999) Transgenic Res. 8: 223-235) from position effects. Much of this activity is contained in a 250-bp fragment. Within this stretch is a 49-bp cHS4 core (Chung, et al. (1997) Proc. Natl. Acad. Sci. USA 94:575-580) that interacts with the zinc finger DNA binding protein CTCF implicated in enhancer-blocking assays (Bell, et al. (1999) Cell 98:387-396).

An illustrative and suitable insulator is FB (FII/BEAD-A), a 77 bp insulator element, that contains the minimal CTCF binding site enhancer-blocking components of the chicken β-globin 5′ HS4 insulators and a homologous region from the human T-cell receptor alpha/delta blocking element alpha/delta I (BEAD-I) insulator described by Ramezani, et al. ((2008) Stem Cell 26:3257-3266). The FB “synthetic” insulator has full enhancer blocking activity. In addition to FB, other suitable insulators include, for example, the full length chicken beta-globin HS4 or insulator sub-fragments thereof, the ankyrin gene insulator, and other synthetic insulator elements.

Packaging Signal.

The lentiviral construct of this invention can further include a packaging signal. A “packaging signal,” “packaging sequence,” or “psi sequence” is any nucleic acid sequence sufficient to direct packaging of a nucleic acid whose sequence includes the packaging signal into a retroviral particle. The term includes naturally occurring packaging sequences and also engineered variants thereof. Packaging signals of a number of different retroviruses, including lentiviruses, are known in the art.

Rev Responsive Element (RRE).

The lentiviral construct of this invention can also include a Rev response element (RRE) to enhance nuclear export of unspliced RNA. RREs are well-known to those of skill in the art. Illustrative RREs include, but are not limited to RREs such as that located at positions 7622-8459 in the HIV NL4-3 genome (GENBANK accession number AF003887) as well as RREs from other strains of HIV or other retroviruses.

Central PolyPurine Tract (cPPT).

In some embodiments, the lentiviral construct described herein further include a central polypurine tract. Insertion of a fragment containing the central polypurine tract (cPPT) in lentiviral (e.g., HIV-1) vector constructs is known to enhance transduction efficiency drastically by facilitating the nuclear import of viral cDNA through a central DNA flap.

Expression-Stimulating Post-Transcriptional Regulatory Element (PRE).

In certain embodiments, the lentiviral construct described herein may further include any of a variety of post-transcriptional regulatory elements (PREs) whose presence within a transcript increases expression of the heterologous nucleic acid at the protein level. PREs may be particularly useful in certain embodiments, especially those that involve lentiviral constructs with modest promoters. One type of PRE is an intron positioned within the expression cassette, which can stimulate gene expression. However, introns can be spliced out during the life cycle events of a lentivirus. Hence, if introns are used as PRE's they are typically placed in an opposite orientation to the vector genomic transcript.

Post-transcriptional regulatory elements that do not rely on splicing events offer the advantage of not being removed during the viral life cycle. Some examples are the post-transcriptional processing element of herpes simplex virus, the post-transcriptional regulatory element of the hepatitis B virus (HPRE) and the woodchuck hepatitis virus (WPRE). Of these the WPRE is typically preferred as it contains an additional cis-acting element not found in the HPRE. This regulatory element is typically positioned within the vector so as to be included in the RNA transcript of the transgene, but outside of stop codon of the transgene translational unit. The WPRE is characterized and described in U.S. Pat. No. 6,136,597.

The recombinant lentiviral construct and resulting virus described herein transfer nucleic acids encoding HMGA2 into a mammalian cell and optionally nucleic acids encoding one or more therapeutic agents. For delivery to cells, vectors of the present invention are preferably used in conjunction with a suitable packaging cell line or co-transfected into cells in vitro along with other vector plasmids containing the necessary retroviral genes (e.g., gag and pol) to form replication incompetent virions capable of packaging the vectors of the present invention and infecting cells.

Typically, the vectors are introduced via transfection into the packaging cell line. The packaging cell line produces viral particles that contain the vector genome. Methods for transfection are well-known to those of skill in the art. After co-transfection of the packaging vectors and the lentiviral vector to the packaging cell line, the recombinant virus is recovered from the culture media and titered by standard methods used by those of skill in the art. In some embodiments, the packaging constructs are introduced into human cell lines by calcium phosphate transfection, lipofection or electroporation, generally together with a dominant selectable marker, such as neomycin, DHFR, glutamine synthetase, followed by selection in the presence of the appropriate drug and isolation of clones. In certain embodiments the selectable marker gene can be linked physically to the packaging genes in the construct.

Stable cell lines, wherein the packaging functions are configured to be expressed by a suitable packaging cell, are known in the art (see, e.g., U.S. Pat. No. 5,686,279, which describes packaging cells). In general, for the production of virus particles, one may employ any cell that is compatible with the expression of lentiviral Gag and Pol genes, or any cell that can be engineered to support such expression. For example, producer cells such as 293T cells and HT1080 cells may be used.

The packaging cells with a lentiviral vector incorporated in them form producer cells. Producer cells are thus cells or cell-lines that can produce or release packaged infectious viral particles carrying the nucleic acids encoding HMGA2 and the therapeutic agent(s) of interest. These cells can further be anchorage-dependent which means that these cells will grow, survive, or maintain function optimally when attached to a surface such as glass or plastic. Some examples of anchorage-dependent cell lines used as lentiviral vector packaging cell lines when the vector is replication competent are HeLa or 293 cells and PERC.6 cells.

Accordingly, in certain embodiments, methods are provided of delivering a gene to a cell, which is then integrated into the genome of the cell, by contacting the cell with a virion containing a retroviral vector described herein. The cell (e.g., in the form of tissue or an organ) can be contacted (e.g., infected) with the virion ex vivo and then delivered to a subject (e.g., a mammal, animal or human) in which the gene (e.g., anti-sickling β-globin) will be expressed. In various embodiments the cell can be autologous to the subject (i.e., from the subject) or it can be non-autologous (i.e., allogeneic or xenogenic) to the subject. Moreover, because the vectors described herein are capable of being delivered to both dividing and non-dividing cells, the cells can be from a wide variety including, for example, bone marrow cells, mesenchymal stem cells (e.g., obtained from adipose tissue), and other primary cells derived from human and animal sources. Alternatively, the virion can be directly administered in vivo to a subject or a localized area of a subject (e.g., bone marrow).

Of course, as noted above, the lentivectors described herein will be particularly useful in the transduction of human hematopoietic progenitor cells or a hematopoietic stem cells, obtained either from the bone marrow, the peripheral blood or the umbilical cord blood, as well as in the transduction of a CD4⁺ T cell, a peripheral blood B or T lymphocyte cell, and the like. In certain embodiments particularly preferred targets are CD34⁺ cells.

Gene Therapy.

In light of the fact that the present retroviral vector was shown to improve efficacy, enhance transduction efficiency, and facilitate expansion of hematopoietic stem cells in vivo, the present invention is also directed to a method for increasing the efficacy and in vivo expansion of cells, in particular human hematopoietic stem cell by introducing a let-7 insensitive nucleic acid encoding a high mobility group AT-hook 2 (HMGA2) protein into a retroviral vector and contacting a population of cells with the retroviral vector encoding the HMGA2 protein under conditions to effect the transduction of a cell in said population by the vector to increase the efficacy and in vivo expansion of the cells. For the purposes of this invention, the phrase “in vivo expansion” is used herein to describe a process of cell proliferation in a manner substantially devoid of cell differentiation. Cells that undergo expansion hence maintain their cell self-renewal properties. Moreover, having demonstrated that a small population (about 1%) of stem cells can expand to between 40 and 70% over a period of two years without causing hematopoietic abnormalities (e.g., leukemia), the retroviral construct can effectively be used to provide long-term delivery of a therapeutic agent to a human subject.

The cells (e.g., stem cells) may be transduced in vivo or in vitro, depending on the ultimate application. Even in the context of human gene therapy, such as gene therapy of human stem cells, one may transduce the stem cell in vivo or, alternatively, transduce in vitro followed by infusion of the transduced stem cell into a human subject. In one aspect of this embodiment, the cell is a stem cell removed from a human, e.g., a human patient, using methods well-known to those of skill in the art and transduced as noted above. The transduced stem cells are then reintroduced into the same or a different human.

The retroviral construct described herein is particularly useful for the transduction of human hematopoietic progenitor cells or hematopoietic stem cells (HSCs), obtained, e.g., from the bone marrow (CD34⁺ cells), the peripheral blood or the umbilical cord blood, as well as in the transduction of a CD4⁺ T cell, a peripheral blood B or T lymphocyte cell, and the like. Examples of adult stem cells that can be transduced using the retroviral construct of this invention and used to obtain the indicated phenotype in a target tissue of interest, are listed in Table 1.

TABLE 1

| | Differentiated | Target | |
|---|---|---|---|
| Stem cell | phenotype | tissue | Reference |
| Bone marrow | Oval cells, | Liver | Petersen (1999) |
| | Hepatocytes | | Science 284: 1168-1170 |
| KTLS cells | Hepatocytes | Liver | Lagasse (2000) Nat. |
| | | | Med. 6: 1229-1234 |
| Bone marrow | Hepatocytes | Liver | Alison (2000) Nature |
| | | | 406: 257; Thiese |
| | | | (2000) Hepatology |
| | | | 32: 11-16 |
| Pancreatic | Hepatocytes | Liver | Shen (2000) Nat. Cell |
| exocrine | | | Biol. 2: 879-887 |
| cells | | | |
| Pancreas | Hepatocytes | Liver | Wang (2001) Am. J. |
| | | | Pathol. 158: 571-579 |
| Bone marrow | Endothelium | Liver | Gao (2001) Lancet |
| | | | 357: 932-933 |
| Bone marrow | Tubular | Kidney | Poulsom (2001) J. |
| | epithelium, | | Pathol. 195: 229-235 |
| | glomeruli | | |
| Bone marrow | Endothelium | Kidney | Lagaaij (2001) Lancet |
| | | | 357:33-37 |
| Extra renal | Endothelium | Kidney | Williams (1969) Surg. |
| | | | Forum 20: 293-294 |
| Bone marrow | Myocardium | Heart | Orlic (2001) Nature |
| | | | 410: 701-704 |
| Bone marrow | Cardiomyocytes | Heart | Jackson (2001) J. |
| | and | | Clin. Invest. |
| | Endothelium | | 107: 1395-1402 |
| Bone marrow | Type 1 | Lung | Krause (2001) Cell |
| | pneumocytes | | 105: 369-377 |
| Neuronal | Multiple | Marrow | Bjornson (1999) |
| | hematopoietic | | Science 283: 534-537 |
| | lineages | | |
| Bone marrow | Neurons | CNS | Mezey (2000) Science |
| | | | 290: 1779-1782 |
| Bone marrow | Microglia and | CNS | Eglitis (1997) Proc. |
| | Astrocyes | | Natl. Acad. Sci. USA |
| | | | 94: 4080-4085. |

SP, Side population cells; CNS, central nervous system.

When cells, for instance CD34⁺ cells, dendritic cells, peripheral blood cells or tumor cells are transduced ex vivo, the vector particles are incubated with the cells using a dose generally in the order of between 1 to 150 or more particularly 10 to 150 multiplicities of infection (MOI) which also corresponds to 1×10⁵ to 50×10⁵ transducing units of the viral vector per 10⁵ cells. This of course includes amount of vector corresponding to 1, 2, 3, 4, 5, 6, 7, 8, 9, 10, 15, 20, 25, 30, 35, 40, 45, and 50 MOI. Typically, the amount of vector may be expressed in terms of HeLa transducing units (TU).

In certain embodiments, cell-based therapies involve providing stem cells and/or hematopoietic precursors, transducing the cells with the retrovirus encoding HMGA2 and one or more therapeutic agents of interest, and then introducing the transformed cells into a subject in need thereof (e.g., a subject with sickle cell anemia or an immunodeficiency). In certain embodiments, a therapeutic method involves isolating population of cells, e.g., stem cells from a subject, optionally expanding the cells in tissue culture, and introducing the retrovirus encoding HMGA2 and one or more therapeutic agents of interest into the cells in vitro. The cells are then returned to the subject, where, for example, they may provide a population of cells that produce HMGA2 and one or more therapeutic agents. In certain embodiments, the population of cells that produce HMGA2 expand by at least 10-fold, 20-fold, 30-fold, 40-fold, 50-fold, 60-fold, 70-fold, or more after introduction into the subject. In other embodiments, the population of transduced cells in maintained in the subject for at least 1 year, 2 years, 3 years, 4 years, 5 years, or more to provide long-term delivery of the one or more therapeutic agents to the subject.

In some embodiments of the invention, a population of cells, which may be cells from a cell line or from an individual other than the subject, can be used. It will be recognized that such cells can be derived from a number of sources including bone marrow (BM), cord blood (CB), mobilized peripheral blood stem cells (mPBSC), and the like. Methods of isolating stem cells, immune system cells, etc., from a subject or from an individual other than the subject and administering them to a subject are well-known in the art. Such methods are used, e.g., for bone marrow transplant, peripheral blood stem cell transplant, etc., in patients undergoing chemotherapy.

In certain embodiments, the let-7 insensitive nucleic acid encoding HMGA2 (i.e., lacking the 3′ let-7 sites) is inserted into a MSCV-IRES lentiviral vector, preferably including nucleic acids encoding a therapeutic agent, and used in stem cell gene therapy. The recombinant lentiviral construct is introduced into the CD34+ cells of patients in need of treatment followed by autologous transplantation.

Direct Introduction of Vector.

In alternative embodiments, treatment of a subject is carried out by direct introduction of the retroviral construct. The retroviral construct may be formulated for delivery by any available route including, but not limited to parenteral (e.g., intravenous), intradermal, interosseous, subcutaneous, oral (e.g., inhalation), transdermal (topical), transmucosal, rectal, or vaginal. Commonly used routes of delivery include inhalation, parenteral, and transmucosal.

For such therapeutic applications, the retroviral construct is typically formulated in combination with a pharmaceutically acceptable carrier. As used herein the phrase “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

Active agents, i.e., a retroviral construct described herein and/or other agents to be administered together the vector, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such compositions will be apparent to those skilled in the art. Suitable materials can also be obtained commercially from Alza Corporation and Nova Pharmaceuticals, Inc.

Liposomes can also be used as pharmaceutically acceptable carriers. These can be prepared according to methods known to those skilled in the art, for example, as described in U.S. Pat. No. 4,522,811. In some embodiments the composition is targeted to particular cell types or to cells that are infected by a virus. For example, compositions can be targeted using monoclonal antibodies to cell surface markers, e.g., CD34 protein.

It is advantageous to formulate compositions in dosage unit form for ease of administration and uniformity of dosage. Dosage unit form as used herein refers to physically discrete units suited as unitary dosages for the subject to be treated; each unit including a predetermined quantity of a retroviral construct calculated to produce the desired therapeutic effect in association with a pharmaceutical carrier.

A unit dose need not be administered as a single injection but may include continuous infusion over a set period of time. Unit dose of the retroviral construct described herein may conveniently be described in terms of transducing units (T.U.) of lentivector, as defined by titering the vector on a cell line such as HeLa or 293. In certain embodiments unit doses can range from 10³, 10⁴, 10⁵, 10⁶, 10⁷, 10⁸, 10⁹, 10¹⁰, 10¹¹, 10¹², 10¹³ T.U. and higher.

Pharmaceutical compositions can be administered at various intervals and over different periods of time as required, e.g., one time per week for between about 1 to about 10 weeks; between about 2 to about 8 weeks; between about 3 to about 7 weeks; about 4 weeks; about 5 weeks; about 6 weeks, etc. It may be necessary to administer the therapeutic composition on an indefinite basis. The skilled artisan will appreciate that certain factors can influence the dosage and timing required to effectively treat a subject, including but not limited to the severity of the disease or disorder, previous treatments, the general health and/or age of the subject, and other diseases present. Treatment of a subject with a retroviral construct can include a single treatment or, in many cases, can include a series of treatments.

Exemplary doses for administration of gene therapy vectors and methods for determining suitable doses are known in the art. It is furthermore understood that appropriate doses of a retroviral construct may depend upon the particular recipient and the mode of administration. The appropriate dose level for any particular subject may depend upon a variety of factors including the age, body weight, general health, gender, and diet of the subject, the time of administration, the route of administration, the rate of excretion, other administered therapeutic agents, and the like.

In certain embodiments, retroviral gene therapy vectors can be delivered to a subject by, for example, intravenous injection, local administration, or by stereotactic injection (see, e.g., Chen, et al. (1994) Proc. Natl. Acad. Sci. USA 91:3054). In certain embodiments, vectors may be delivered via into the bone marrow cavity and may be encapsulated or otherwise manipulated to protect them from degradation, enhance uptake into tissues or cells, etc. Pharmaceutical preparations can include a retroviral construct in an acceptable diluent, or can include a slow release matrix in which a retroviral construct is imbedded. Alternatively or additionally, where a vector can be produced intact from recombinant cells, as is the case for retroviral or lentiviral vectors as described herein, a pharmaceutical preparation can include one or more cells that produce vectors. Pharmaceutical compositions including a lentiviral construct described herein can be included in a container, pack, or dispenser, e.g., in a kit, optionally together with instructions for administration.

Gene therapy is the modification of the nucleic acid content of cells for therapeutic purposes. While early clinical gene therapy successes were limited, there have been a number of successful clinical gene therapy trials. These include the restoration of vision to patients with Leber's Congenital Amaurosis (LCA) with an AAV vector (Maguire, et al. (2008) N. Engl. J. Med. 358(21):2240-8; Simonelli, et al. (2010) Mol. Ther. 18:643-50; Jacobson, et al. (2015) N. Engl. J. Med. 372:1920-6; Bainbridge, et al. (2015) N. Engl. J. Med. 372:1887-97; Testa, et al. (2013) Ophthamol. 120:1283-91), the generation of therapeutic factor IX levels from in vivo AAV transduction of liver for hemophilia B (Kay, et al. (2000) Nat. Genet. 24(3):257-61; Manno, et al. (2006) Nat. Med. 12(3):342-7; Nathwani, et al. (2011) N. Engl. J. Med. 365(25):2357-65), the remission of leukemia and B-cell lymphoma through the lentiviral transduction of T-cells with a chimeric antigen receptor against CD19 (Porter, et al. (2011) N. Engl. J. Med. 365(8):725-33; Kochenderfer, et al. (2015) J. Clin. Oncol. 33:540-9; Brentjens, et al. (2013) Sci. Transl. Med. 5:177ra38; Maude, et al. (2014) N. Engl. J. Med. 371:1507-17; Lee, et al. (2015) Lancet 385:417-28), the restoration of a functional immune system by ex vivo retroviral transduction of hematopoetic stem and progenitor cells for the primary immunodeficiencies SCID-X1, ADA-SCID, and Wiskott-Aldrich syndrome (WAS) (Aiuti, et al. (2002) Science 296(5577):2410-3; Hacein-Bey-Abina, et al. (2014) N. Engl. J. Med. 371:1407-17; Blaese, et al. (1995) Science 270(5235):475-80; Bortug, et al. (2010) N. Engl. J. Med. 363(20):1918-27; Cavazzana-Calvo, et al. (2000) Science 288(5466):669-72; Hacein-Bey Abina, et al. (2015) J. Am. Med. Assoc. 313:1550-63), the establishment of transfusion independence of a β-thalassemia patient after the ex vivo transduction of hematopoietic stem and progenitor cells with a lentiviral vector (Cavazzana-Calvo, et al. (2010) Nature 467(7313):318-22), restoration of adenosine triphosphate-binding cassette transporter (ALD protein) expression after ex vivo lentiviral transduction of CD34⁺ cells for Adrenoleukodystrophy (Cartier, et al. (2009) Science 326:818-23; Cartier, et al. (2012) Meth. Enzymol. 507:187-98), reconstitution of arylsulfatase A expression by ex vivo lentiviral transduction of CD34⁺ cells for Metachromatic leukodystrophy (Biffi, et al. (2013) Science 341:1233158). Therefore, the lentiviral construct of this invention can be used as an alternative delivery vector in one or more of these diseases to improve transfection efficiency and hematopoietic stem cell expansion.

In addition to the transduction of wild-type hematopoietic stem cells, the present invention also embraces the transduction of hematopoietic stem cells that have been modified by genome editing for therapeutic purposes. Genome editing refers to the process of altering the expression or correcting one or more genes encoding proteins involved in a genetic disease (e.g., producing proteins lacking, deficient or aberrant in the disease and/or proteins that regulate these proteins) such as sickle cell disease or a thalassemia. Such alterations or corrections can result in the treatment of these genetic diseases. However, allele targeting in HSCs is low using current genome editing techniques, in particular homology-directed-repair, because such techniques are inefficient in non-dividing HSCs. Therefore, introduction of a HMGA2 construct during a genome editing procedure will result in HSC amplification in vivo and enhance genome editing efficiency. By way of illustration, genome editing can be used to correct the β-globin allele for sickle cell disease with concurrent introduction of HMGA2 so that the hematopoietic stem cells containing the appropriate editing event can be expanded over time.

Accordingly, the present invention also provides a genetically-modified hematopoietic stem cell harboring a genetic alteration incurred by genome editing and including a construct having a let-7 insensitive nucleic acid encoding a HMGA2 protein. The let-7 insensitive nucleic acid encoding the HMGA2 protein can be introduced into the hematopoietic stem cell using the retroviral construct of the present invention or by inclusion of HMGA2 nucleic acid in the homology template used for correcting the locus of interest. As used herein, genome editing refers to the process whereby one or more endonuclease(s) or endonuclease fusion protein(s) are introduced into a cell to achieve targeted gene modification and/or disruption. Four protein scaffolds are known in the art for targeted gene modification and/or disruption. These include zinc finger nucleases (ZFNs), transcription activator-like effector (TALE) nucleases (TALENs), homing endonucleases (HEs), and clustered regularly interspaced short palindromic repeats (CRISPR) and CRISPR-associated (Cas) systems in combination with a RNA guide strand.

ZFNs are fusion proteins composed of an array of site-specific DNA-binding domains, which are adapted from zinc finger-containing transcription factors, attached to the endonuclease domain of the bacterial FokI restriction enzyme. Each zinc finger domain recognizes a 3- to 4-bp DNA sequence, and tandem domains can potentially bind to an extended nucleotide sequence (typically with a length that is a multiple of 3, usually 9 bp to 18 bp) that is unique within a cell's genome. To cleave a specific site in the genome, ZFNs are designed as a pair that recognizes two sequences flanking the site, one on the forward strand and the other on the reverse strand. Upon binding of the ZFNs on either side of the site, the FokI domains dimerize and cleave the DNA at the site, generating a double-strand break (DSB) with 5′ overhangs (Urnov, et al. (2010) Nat. Rev. Genet. 11(9):636-646). Cells repair DSBs using either (a) nonhomologous end joining (NHEJ), which can occur during any phase of the cell cycle, but occasionally results in erroneous repair, or (b) homology-directed repair (HDR), which typically occurs during late S phase or G₂ phase when a sister chromatid is available to serve as a repair template. The error-prone nature of NHEJ can be exploited to introduce frame-shifts into the coding sequence of a gene, potentially knocking out the gene by a combination of two mechanisms: premature truncation of the protein and nonsense-mediated decay of the mRNA transcript. Alternatively, HDR can be utilized to insert a specific mutation, with the introduction of a repair template containing the desired mutation flanked by homology arms. In response to a DSB in DNA, HDR uses another closely matching DNA sequence to repair the break. Mechanistically, HDR can proceed in the same fashion as traditional homologous recombination, using an exogenous double-stranded DNA vector as a repair template (Rouet, et al. (1994) Proc. Natl. Acad. Sci. USA 91(13):6064-6068). It can also use an exogenous single-stranded DNA oligonucleotide (ssODN) as a repair template. For ssODNs, homology arms of as little as 20 bp can enable introduction of mutations into the genome (Radecke, et al. (2010) Mol. Ther. 18:743-53; Soldner, et al. (2011) Cell 146:318-31; Chen, et al. (2011) Nat. Methods 8:753-5).

TALENs are a class of proteins containing TALE repeats. The naturally occurring TALE repeats are composed of tandem arrays with 10 to 30 repeats that bind and recognize extended DNA sequences (Bogdanove & Voytas (2011) Science 333(6051):1843-1846). Each repeat is 33 to 35 amino acids in length, with two adjacent amino acids (termed the repeat-variable di-residue (RVD)) conferring specificity for one of the four DNA base pairs (Moscou & Bogdanove (2009) Science 326:1501; Boch, et al. (2009) Science 326:1509-12; Morbitzer, et al. (2010) Proc. Natl. Acad. Sci. USA 107:21617-22)). Thus, there is a one-to-one correspondence between the repeats and the base pairs in the target DNA sequences. TALENs are generated by engineering many TALE repeat arrays that bind with high affinity to desired genomic DNA sequences. TALENs are often designed to bind 18-bp sequences or even longer and, when fused to the FokI endonuclease domain, can generate DSBs at a desired target site in the genome thereby knocking out genes or knocking in mutations.

HEs are small proteins (<300 amino acids) found in bacteria, archaea, and in unicellular eukaryotes. A distinguishing characteristic of HEs is that they recognize relatively long sequences (14-40 bp) compared to other site-specific endonucleases such as restriction enzymes (4-8 bp). At least five such families have been identified: LAGLIDADG; GIY-YIG; HNH; His-Cys Box and PD-(D/E)xK, which are related to EDxHD enzymes. HEs can be used for insertion, deletion, single-site mutation, and correction, in a highly site-specific and controlled fashion. See Belfort & Bonocora (2014) Methods Mol. Biol. 1123:1-26.

CRISPR-Cas systems use a combination of proteins and short RNAs to target specific DNA sequences for cleavage. Expression of the Cas9 protein along with guide RNA(s) (either two separate RNAs, as found in bacteria, or a single chimeric RNA), in mammalian cells results in DSBs at target sites with (a) a 20-bp sequence matching the protospacer of the guide RNA and (b) an adjacent downstream NGG nucleotide sequence (termed the protospacer-adjacent motif (PAM)(Cong, et al. (2013) Science 339:819-23; Mali, et al. (2013) Science 339:823-6; Jinek, et al. (2012) Science 337:816-21; Cho, et al. (2013) Nat. Biotechnol. 31:230-2). This occurs via the formation of a ternary complex in which Cas9 binds to the PAM in the DNA, then binds the nonprotospacer portion of the guide RNA, upon which the protospacer of the guide RNA hybridizes with one strand of the genomic DNA. Cas9 then catalyzes the DSB in the DNA at a position 3 bp upstream of the PAM. CRISPR-Cas9 can be easily adapted to target any genomic sequence by changing the 20-bp protospacer of the guide RNA, which can be accomplished by subcloning this nucleotide sequence into the guide RNA plasmid backbone. The Cas9 protein component remains unchanged.

A genetically-modified hematopoietic stem cell of this invention is produced by introducing genome editing components along with a let-7 insensitive nucleic acid encoding a HMGA2 protein into a hematopoietic stem cell (e.g., an autologous HSC). In some embodiments, the genome editing components are introduced into the cell by the retroviral construct described herein. In other embodiments, the genome editing components are introduced into the cell by a separate vector. In further embodiments, the let-7 insensitive nucleic acid encoding a HMGA2 protein is included in the template used for homology directed repair. Once produced, the genetically-modified hematopoietic stem cell can be introduced into a subject to provide clinical benefit. In particular, it is posited that the let-7 insensitive nucleic acid encoding the HMGA2 protein will facilitate in vivo expansion thereby enhancing genome editing efficiency to correct an aberrant gene, insert a wild-type gene, or change the expression of an endogenous gene in a subject in need of treatment. By way of illustration, genome editing can be used to disrupt a fetal hemoglobin (HbF) silencing DNA regulatory element or pathway; mutate one or more γ-globin gene promoter(s) to achieve increased expression of a γ-globin gene; mutate one or more δ-globin gene promoter(s) to achieve increased expression of a δ-globin gene; and/or correct one or more β-globin gene mutation(s) to treat hemoglobinopathies such as thalassemias and sickle-cell disease. This strategy can be combined with inclusion of an HMGA2 let-7 insensitive expression construct that is stably integrated into the HSCs containing the HbF-producing editing event. Indeed, HMGA2 has been found to modulate myelo-erythropoietic developmental decisions in mice (Rowe, et al. (2016) J. Exp. Med. 213(8):1497-512). This analysis indicates that increased HMGA2 skews development toward erythroid-dominant erythropoiesis. Further, HMGA2 over-expression provides an increase in fetal hemoglobin (de Vasconcellos, et al. (2016) PLoS ONE 11(11):e0166928), thereby demonstrating that the instant HMGA2 protein is useful in the treatment of hemoglobin disorders.

Serious hematologic malignancies are typically treated through high dose or lethal chemotherapy and/or radiation therapy conditioning regimens followed by rescue with allogeneic stem cell transplantation (allo-SCT) or autologous stem cell transplantation (ASCT). These myeloablative/lymphoablative (M/L) treatment regimens involve the elimination of both the patient's hematopoietic stem cells and T-lymphocytes by cell killing, blocking, and/or down-regulation, of substantially all the hematopoietic stem cells and lymphocytes of the patient. Patients treated by allo-SCT or ASCT can develop major complications due to the M/L conditioning. In addition, patients receiving allo-SCT are susceptible to graft versus host disease (GVHD), as well as to graft rejection. Moreover, relapse is still a frequent problem in these patients. In light of the enhanced expansion capacity of the HMGA2-transduced stem cells described herein, these cells can be used to reduce the amount of potentially toxic, myeloablative conditioning used in a patient receiving the therapeutically modified HSCs. For example, reduction of myelosuppressive conditioning would normally result in a decrease in the number of genetically-modified HSCs that engraft in the recipient. The use of the retroviral construct of this invention would permit the use of lower dose and safer conditioning regimens because otherwise suboptimal levels of HSC engraftment could be therapeutically sufficient if subsequently followed by HMGA2-mediated expansion of those few HSCs that did engraft. Thus, in certain embodiments of this invention, a subject being treated in accordance with the methods herein, receives a reduced intensity or low dose myeloablative conditioning regime. Examples of “high dose” conditioning regimens include 12-16 Gy total body irradiation in combination with a chemotherapeutic agent such as cyclphosphamide (120 mg/kg), cytarabine, etoposide, melphalan or busulfan or combinations of chemotherapeutic agents such as 16 mg/kg busulfan and 200 mg/kg cyclophosphamide or 140 mg/m² melphalan. By comparison, “low dose” or reduced intensity myeloablative conditioning regimes include, but are not limited to, the use of a purine analog (e.g., fludarabine or cladribine) in combination with melphalan (e.g., 180, 140, or 100 mg/m²)(Oran, et al. (2007) Biol. Blood Marrow Transplant 13:454-62; Popat, et al. (2012) Bone Marrow Transplant. 47:212-6); fludarabine, oral busulfan (8 mg/kg), and anti-thymocyte globulin (ATG)(Slavin, et al. (1998) Blood 91:756-63); fludarabine, cytarabine, and amsacrine, followed by 3 days of rest, and then 4-Gy TBI, ATG, and cyclophosphamide (80-120 mg/kg; the fludarabine, AraC, amsacrine regimen)(Schmid, et al. (2005) J. Clin. Oncol. 23:5675-87).

The following non-limiting examples are provided to further illustrate the present invention.

### Example 1: HMGA2-Induced Clonal Expansion in a Non-Human Primate Stem Cell Transplant Model

The human HMGA2 cDNA, without the 3′ let-7 sites, was cloned from 293T cells into a c120c MSCV-IRES-GFP plasmid, which included a gamma-retroviral MSCV promoter along with an IRES-GFP cassette (i.e., LTR-MSCV-HMGA2-IRES-GFP-LTR). Lentiviral HMGA2-GFP was produced with a typical unconcentrated titer between 5e7 and 1e8 to/mL. HMGA2 expression was verified with flow cytometry and western blot analyses in primate CD34+ cells and multiple human cell lines, respectively. For transplant studies, GCSF-mobilized bone marrow was harvested from two macaques, A10W027 and A10W016. Bone marrow was enriched for CD34+ cells and stimulated overnight on RETRONECTIN-coated plates in XVIVO10 media containing human serum albumin (HSA), stem cell factor (SCF), Fms-related tyrosine kinase 3 ligand (Flt3-L), and thrombopoietin (TPO). The CD34⁺ cells were then split in half and each received two vector exposures, HMGA2-GFP vector (MOI=50) and mCherry vector (i.e., LTR-MSCV-IRES-mCherry-LTR)(MOI=100), with an 8 hour washout between applications.

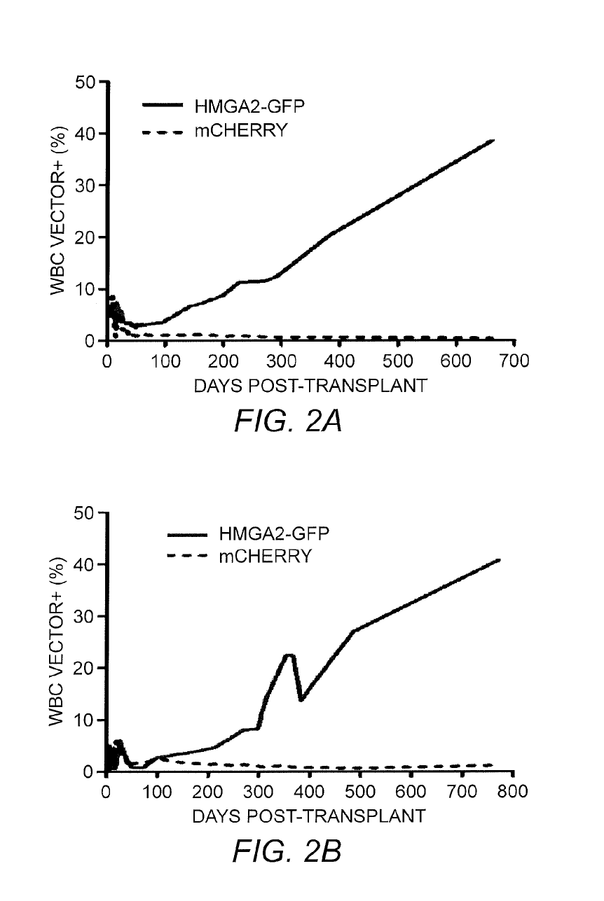

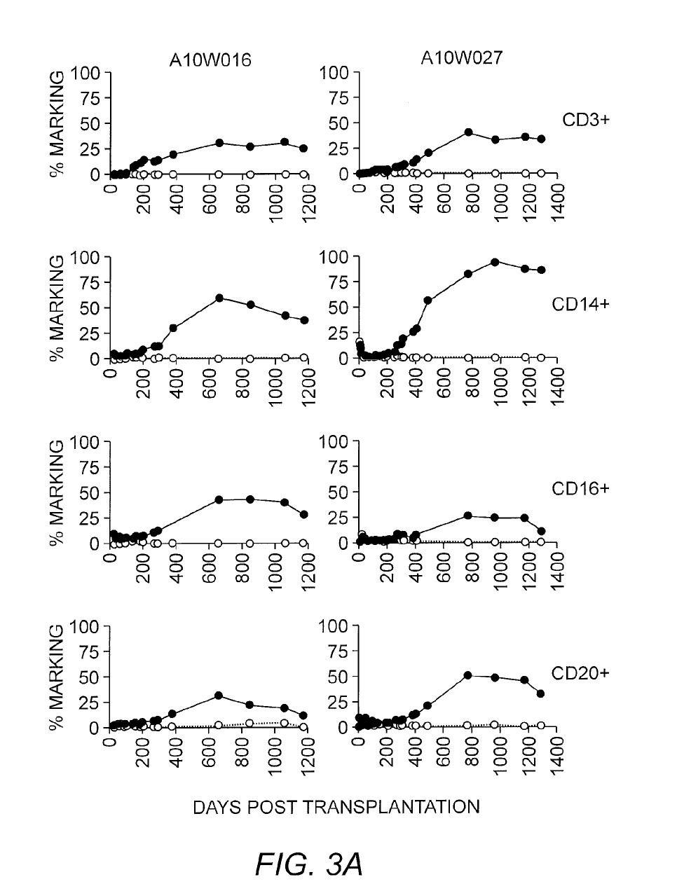

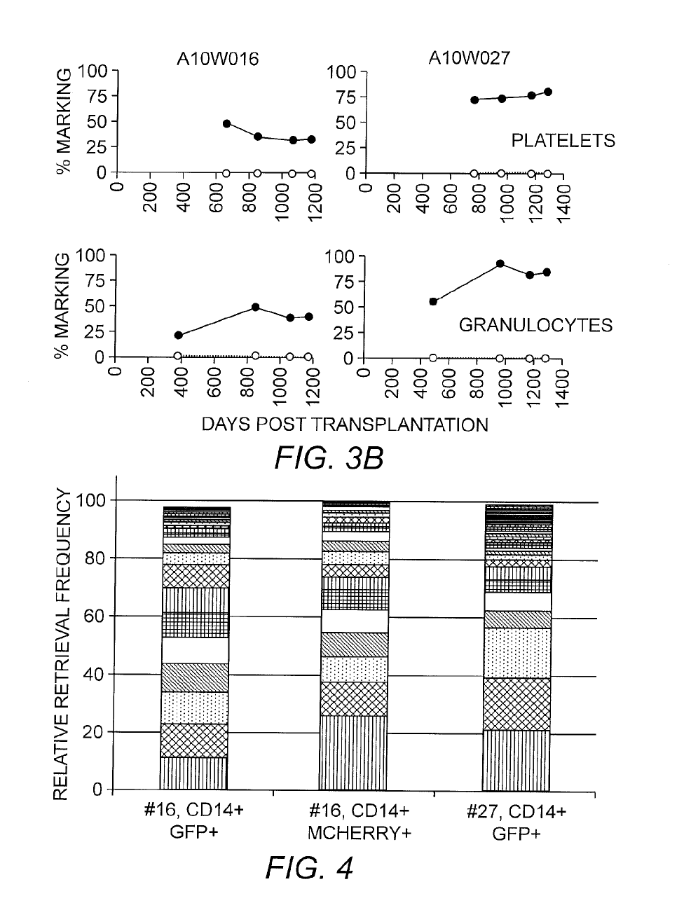

After transduction, autologous cells were mixed together in a 50:50 ratio to form a split graft of HMGA2-GFP and mCherry cells. Two animals (#16 and #27) were transplanted following lethal total body irradiation (475 rads×2) and studied for 21 to 26 months after transplant. The transduction efficiency in the total graft for animal #16 was 42.9% for GFP and 19.1% for mCherry, and for animal #27 was 6% for GFP and 7.9% for mCherry. At three months post-transplantation, the marking in the peripheral blood mononuclear cells was 2.9% GFP⁺ and 1.1% mCherry⁺ for animal #16, and was 2.7% GFP⁺ and 3.2% mCherry⁺ for animal #27, indicating relatively equivalent and low levels of transduction of HSCs and progenitors with each of the two vectors. The HMGA2-GFP marking progressively increased over 21 and 26 months in the peripheral blood leukocytes to 39% for #16 and 41% for #27 while the mCherry marked cells have decreased (FIGS. 2A and 2B, respectively). Equivalent levels of marking were seen in various mature peripheral blood lineages (CD3⁺ T cells, CD14⁺ myeloid cells, CD16⁺ NK cells, CD20⁺ B cells, platelets and granulocytes) at 1200 days after transplant, indicating that expansion had occurred in pluripotent HSCs (FIGS. 3A and 3B). This HSC expansion was further demonstrated by a marking analysis in bone marrow cells that showed about 50% and about 80% GFP marking in the CD34⁺CD45RA⁻ HSC compartment for animal #16 and #27 respectively, at the latest time point. By comparison marking from the control vector in these monkeys was less than 1%.

Clonality analyses using vector integration sites (VIS) at 10.5 months showed overall oligoclonal marking in both the GFP⁺ and mCherry⁺ cells in both animals, with numerous clones contributing to the peripheral blood CD14⁺ compartment (FIG. 4). The top most frequent VISs were present in all peripheral blood lineages (Table 2) indicating that expansion had occurred in multiple HSC clones. This pattern of clonality stably persisted up to the most recent analysis. The white blood cell counts, lineage distribution in the peripheral blood, the percentage of CD34⁺ cells in the bone marrow and all the mature lineages in the peripheral blood were all within the normal range, demonstrating lack of any detectable hematopoietic abnormality.

TABLE 2

| A10W016 | | | | A10W027 | | | |
|---|---|---|---|---|---|---|---|
| mCherry⁺ | | HMGA2-GFP⁺ | | mCherry⁺ | | HMGA2-GFP+ | |
| Location | % | Location | % | Location | % | Location | % |
| (Human) | Reads | (Human) | Reads | (Human) | Reads | (Human) | Reads |
| C3orf58 | 57.19 | HSF1 | 62.42 | n/a | 8.95 | DPYD | 4.66 |
| ANKH | 22.28 | NCKAP1L | 27.42 | GTF2E2// | 4.87 | PRRC2A | 4.27 |
| | | | | SMIM18 | | | |
| KDM1B | 7.45 | Intergenic | 3.76 | ZNF41 | 3.28 | Intergenic | 3.76 |
| LILRA6 | 2.85 | MYEF2 | 1.79 | MLLT1 | 2.60 | TRAF2 | 3.56 |
| Intergenic | 1.55 | STXBP5 | 0.92 | PACS1 | 2.20 | BIN2 | 3.47 |
| Intergenic | 1.53 | RTEL1// | 0.28 | CDC42BPG | 1.89 | SLC27A1 | 2.41 |
| | | RTEL1- | | | | | |
| | | TNFRSF6B | | | | | |
| AP2A2 | 0.67 | n/a | 0.28 | PLEC | 1.84 | ASPSCR1 | 2.36 |
| HSF1 | 0.50 | Intergenic | 0.27 | LMNA | 1.53 | Intergenic | 1.73 |
| RBM6 | 0.50 | PLD3 | 0.21 | PRDM16 | 1.26 | Intergenic | 1.71 |
| LRRIQ1 | 0.38 | SOCS7 | 0.19 | MIR548W// | 0.96 | EPS15L1 | 1.18 |
| | | | | TANC2 | | | |

Gene expression microarray analysis of RNA from sorted bone marrow CD34⁺GFP⁺ and CD34⁺mCherry⁺ cells showed sharp upregulation of several genes in the HMGA2-expressing cells, particularly the IGF2BP2 gene, a known downstream target of HMGA2.

Therefore, these data show that long-term HSCs from non-human primates can be progressively expanded in vivo over several years by overexpressing HMGA2 in a large animal model without causing malignancies. Accordingly, the retroviral construct of this invention is useful for gene therapy, particularly for expanding and obtaining high numbers of transduced erythrocytes and granulocytes for diseases in which a naturally occurring selection advantage is not present.

What is claimed is:

1. A retroviral construct comprising a let-7 insensitive nucleic acid encoding a high mobility group AT-hook 2 (HMGA2) protein and nucleic acids encoding, one or more therapeutic agents.

2. The retroviral construct of claim 1, wherein the one or more therapeutic agents comprise a therapeutic protein or nucleic acid.

3. The retroviral construct of claim 1, wherein the let-7 insensitive nucleic acid comprises mutation or deletion of one or more let-7 binding sites.

4. A cell transduced with the retroviral construct of claim 1.

5. The cell of claim 4, wherein said cell is a hematopoietic stem cell.

6. A method for increasing the efficacy and in vivo expansion of transduced cells comprising introducing a let-insensitive nucleic acid encoding a high mobility group AT-hook 2 (HMGA2) protein into a retroviral vector and transducing cells with the retroviral vector encoding the HMGA2 protein to increase the efficacy and in vivo expansion of the cells.

7. The method of claim 6, wherein the retroviral vector further comprises nucleic acids encoding one or more therapeutic agents.

8. The method of claim 6, wherein the let-7 insensitive nucleic acid comprises mutation or deletion of one or more let-7 binding sites.

9. The method of claim 6, wherein the cells are hematopoietic stem cells.

10. A method for treating a disease or condition comprising transducing cells with a retroviral vector having a let-7 insensitive nucleic acid encoding a high mobility group AT-hook 2 (HMGA2) protein and nucleic acids encoding one or more therapeutic agents and introducing the transduced cells into a subject in need of treatment with the one or more therapeutic agents thereby treating the subject's disease or condition.

11. The method of claim 10, wherein the one or more therapeutic agents comprise a therapeutic protein or nucleic acid.

12. The method of claim 10, wherein the let-7 insensitive nucleic acid comprises mutation or deletion of one or more let-7 binding sites.

13. The method of claim 10, wherein the cells are hematopoietic stem cells.

14. The method of claim 10, wherein the subject receives a reduced intensity or low dose myeloablative conditioning regime prior to introducing the transduced cells.

15. A genetically-modified hematopoietic stem cell harboring a genetic alteration incurred by genome editing and including a construct having a let-7 insensitive nucleic acid encoding a high mobility group AT-hook 2 (HMGA2) protein.

16. The genetically-modified hematopoietic stem cell of claim 15, wherein the let-7 insensitive nucleic acid comprises mutation or deletion of one or more let-7 binding sites.

17. A method for enhancing genome editing efficiency comprising introducing into a hematopoietic stem cell harboring a genome editing construct, a construct containing a let-7 insensitive nucleic acid encoding a HMGA2 protein thereby promoting expansion of the hematopoietic stem cell and enhancing genome editing efficiency.

18. A method for treating a disease or condition comprising transducing hematopoietic stem cell with a genome editing construct and a construct comprising a let-7 insensitive nucleic acid encoding a high mobility group AT-hook 2 (HMGA2) protein; and introducing the transduced hematopoietic stem cells into a subject in need of treatment thereby treating the subject's disease or condition.

19. The method of claim 18, wherein the subject receives a reduced intensity or low dose myeloablative conditioning regime prior to introducing the transduced hematopoietic stem cells.
